(19) United States
(12) Patent Application Publication
(10) Pub. No.: US 2017/0114415 A1
(43) Pub. Date: Apr. 27, 2017

(54) ACTIVATING NTRK1 GENE FUSIONS PREDICTIVE OF KINASE INHIBITOR THERAPY

(71) Applicant: THE REGENTS OF THE UNIVERSITY OF COLORADO, a body corporate, Denver, CO (US)

(72) Inventors: Robert C. DOEBELE, Denver, CO (US); Marileila Varella GARCIA, Greenwood Village, CO (US); Anh T. LEE, Denver, CO (US)

(21) Appl. No.: 15/314,760

(22) PCT Filed: Jun. 1, 2015
(86) PCT No.: PCT/US15/33528

§ 371 Date: Nov. 29, 2016

Related U.S. Application Data
(60) Provisional application No. 62/005,734, filed on May 30, 2014.

(51) Int. Cl. C12Q 1/68

(52) U.S. Cl. CPC C12Q 1/6886; C12Q 2600/106; C12Q 2600/158

(57) ABSTRACT

Disclosed are markers, methods and assay systems for the identification of patients suspected of having cancer and/or cancer patients who are predicted to respond, or not respond to the therapeutic administration of specific chemotherapeutic regimens. Particularly, the invention provides a testing paradigm based on tumor cell samples to select cancer patients who will benefit from chemotherapy including one or more kinase inhibitor(s), as well as a paradigm to select cancer patients who will not benefit from such chemotherapy regimen.

### GOVERNMENT INTEREST

This invention was made with Government support under CCSG grant number P30-CA46934 awarded by the National Institutes of Health (NIH), National Cancer Institute (NCI). The US Government has certain rights in this invention.

### FIELD OF THE INVENTION

The present invention generally relates to markers, methods and assay kits for the identification of lung cancer patients predicted to respond to specific cancer therapies.

### BACKGROUND OF THE INVENTION

New anti-cancer compounds that fight tumors may be one of the greatest medical advances in decades keeping some cancer patients in remission for years. But the truth is that this happens for only a minority of patients. Many of these drugs cost $150,000 per year, per patient—even more for higher doses used in some cases—and the health system of many countries is eventually expected to spend billions or even tens of billions of dollars on these drugs each year. Clinicians don't want to give these drugs to 100 percent of the patients if only a small percentage of the patients, will benefit from their administration. Thus, there is a new imperative to develop a test for biomarkers that can identify in advance which patients will benefit from these therapies, sparing others the cost and possible side effects. Such predictive tests would make these new and expensive therapies easier on patient's finances and on the budgets of the national health system of many countries.

The identification of dominant oncogenic mutations, and our ability to specifically inhibit these genetic abnormalities with targeted inhibitors has altered the therapeutic approach for many cancer patients. Activating point mutations, in-frame insertions/deletions, gene amplification, and gene rearrangements can serve as such predictive biomarkers for oncogene-targeted therapies and thus help select patients that have a high likelihood of benefiting from a particular therapy.

In the paradigm of precision oncology, cancer patients are selected for therapy using such predictive biomarkers, rather than using empiric chemotherapy. For example, many of the actionable or potentially actionable oncogenes that represent molecular subtypes in non small cell lung cancer (NSCLC) involve genomic rearrangements with genes encoding receptor tyrosine kinases (RTKs) such as ALK, ROS1, RET, and most recently NTRK1. The study of these low frequency oncogenes not only applies to NSCLC, but is also directly relevant to the treatment of numerous other cancer types: ALK, ROS1, RET, and NTRK1 gene rearrangements have also been observed in other malignancies, expanding the relevance of mutations to colorectal cancer, thyroid cancer, cholangiocarcinoma, glioblastoma, inflammatory myofibroblastic tumors (IMT), ovarian cancer and others. It was estimated in 2007 that gene fusions were reported in approximately 20% of all cancers accounting for a significant proportion of cancer morbidity and mortality (Mitelman F, et al., Nature Reviews Cancer. 2007; 7:233-45). The emergence of high-throughput genomics technologies and programmatic sequencing efforts such as the Cancer Genome Atlas Network and the Cancer Genome Project have generated the molecular profiles of numerous cancers, and this emergent technology has enabled the identification of many additional gene fusions that are putative oncogenes and predicted to be conserved as drivers across breast, glioblastoma, lung, colorectal cancer, and others tumors.

NTRK1 encodes the “high affinity nerve growth factor receptor” also known as the TrkA receptor tyrosine kinase, a member of the Trk (tropomyosin-receptor kinase) family of RTKs that includes the highly homologous TrkB (encoded by NTRK2) and TrkC (encoded by NTRK3) proteins. TrkA, B, and C play important roles in nervous system development through their regulation of cell proliferation, differentiation, apoptosis, and survival of neurons in both the central and peripheral nervous systems. TrkA is activated by its preferred ligand, the nerve growth factor (NGF), but can be activated by the lower affinity ligand NT-3. The binding of NGF to TrkA, induces receptor homodimerization and autophosphorylation of five tyrosine (Y) residues (Y496, Y676, Y680, Y681, and Y791), and these sites serve as phosphorylation-dependent binding sites for various adaptor proteins that contain SH2 or PTB domains, including SHC1, FRS2, GRB2, PI3K, IRS-1, IRS-2, and PLCγ. Once activated, the three Trk family members signal through several downstream signaling pathways including MAPK, AKT, PKC, and NF-Kappa-B, depending on which docking protein(s) bind to the critical phosphorylated tyrosines Y496, Y757, and Y791 (19). Activation of these signaling cascades results in transcriptional and other cell programs that mediate cellular proliferation, synaptic plasticity, neurite outgrowth and repair, prevention or repair of neurodegeneration, sensory neuron maintenance, or apoptosis. It is expected most Trk fusions would employ many or all of the same downstream signalling cascades as the full-length receptors given the preservation of the kinase domain and the critical tyrosine docking sites. The ETV6-NTRK3 fusion might be an exception, because it lacks the critical Y845 docking site for the preferential adaptor SHC1 due to the location of the breakpoint in the fusion and suggests the use of an alternate adaptor, likely IRS-1. Cell type context and differential subcellular localization of fusions might alter the signaling program of the oncogenic fusion kinases.

Loss of normal regulation of TrkA, B or C receptor activity can result in numerous human diseases. Trk receptors are known for mediating pain sensation and can play a role in chronic pain. TrkA loss-of-function mutations are seen in class IV hereditary sensory and autonomic neuropathies (HSAN), such as the genetic disorder congenital insensitivity to pain with anhidrosis (CIPA).

Mutations in Trk family members have been reported in numerous malignancies, including ovarian cancer, colorectal cancer, melanoma, and lung cancer, but only an in-frame deletion of NTRK1 (ΔTrkA) in acute myeloid leukemia (AML) and a splice variant of NTRK1 (TrkAIII) in neuroblastoma have been functionally characterized as oncogenic to date. The most common mechanism of oncogenic activation of TrkA is through genomic rearrangement, and the creation of a gene fusion (Greco A, et al., Mol Cell Endocrinol. 2010; 321:44-9). Interestingly, all of these different mechanisms of oncogenic activation of TrkA (gene rearrangements, deletion, and splice variant) contain the loss of some of the extracellular domain of TrkA, suggesting an unknown regulatory domain in the extracellular domain of TrkA that when lost, results in constitutive activation of the kinase domain and thus its oncogenic capacity. This mechanism is consistent with crystal structure comparison of select RTKs that contain auto inhibitory domains in the extracellular region.

The induction of an autocrine loop involving TrkA and NGF is associated with pro-tumorigenic activity in both breast and prostate carcinoma; similarly, TrkB and BDNF have been shown to play a pro-tumorigenic role in several malignancies, such as breast and prostate cancers. TrkA can act as a tumor suppressor in the absence of cognate ligands. Therefore, expression of these two proteins in the absence of ligand might result in a better prognosis for the patient. As Trk fusions are constitutively activated, they would be expected to be pro-tumorigenic as they are not dependent on ligand for activation.

The typical gene structure for oncogenic fusions is that the 3′ region of a proto-oncogene encoding the kinase-domain is juxtaposed to 5′ sequences from an unrelated gene via an intra- or inter-chromosomal rearrangement. The resultant novel oncogene is both aberrantly expressed and has constitutive activation of the kinase domain. In 1982, the same year that the BCR and ABL genes were implicated in the first oncogenic translocation on the Philadelphia chromosome in chronic myelogenous leukemia (CML), the first NTRK1 gene fusion was identified in a colon cancer sample and contained sequences from TPM3 (non-muscle tropomyosin) (Pulciani S, et al., Nature. 1982; 300:539-42; de Klein A, et al., Nature. 1982; 300:765-7). The incidence and therapeutic potential of TPM3-NTRK1 in colorectal cancer was recently revisited after 32 years by Isacchi and colleagues, reaffirming that this NTRK1 fusion is indeed a reoccurring, albeit infrequent, oncogene in colon cancer (Ardini E, et al., Mol Oncol. 2014.). All four of the colorectal cases harboring NTRK1 fusions identified thus far express the TPM3-NTRK1 oncogene, suggesting a preference for TPM3 as the partner gene in this particular tissue, similar to EML4 with ALK in lung cancer. Chromosomal rearrangements have been observed between NTRK1 and TFG, TPM3, or TPR in papillary thyroid carcinoma (PTC), the most common malignancy of the thyroid (Greco A, et al., Mol Cell Endocrinol. 2010; 321:44-9). Interestingly, many of these activating 5′ gene fusion partners are promiscuous amongst various kinase fusion classes.

Thus, there continues to be a need in the oncology field for the identification of further molecular markers, including NTRK1 oncogenic fusion markers, to facilitate more effective detection and reliably predictive treatment of cancer.

### SUMMARY OF THE DISCLOSURE

The present invention is based on the discovery of gene fusions comprising the NTRK1 gene that are indicative of cancer and also indicative of which patients may respond to cancer therapy comprising therapeutic administration of anti-cancer compounds, such as tyrosine kinase inhibitors.

Accordingly, in one embodiment, the invention comprises a method for determining if a cancer patient is predicted to respond to the administration of a chemotherapeutic regimen. The method comprises detecting in a sample of tumor cells from the patient the presence or absence of a marker, wherein the marker comprises a gene fusion selected from at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1 and wherein the presence or absence of the marker is indicative of whether the cancer patient will respond to the administration of the chemotherapeutic regimen.

In various embodiments, the chemotherapeutic regimen may include administration of one or more of the following: a tyrosine kinase inhibitor, a HSP90 inhibitor (or other chaperone inhibitor), an inhibitor that targets tyrosine kinase downstream signalling cascade, or combinations thereof. Such inhibitors are well known in the art and are commercially available. All such inhibitors are encompassed in the present invention. For instance, in some embodiments, the tyrosine kinase inhibitor may be a TrkA inhibitor. Examples of tyrosine kinase inhibitors include, but are not limited to, gefitinib, erlotinib, crizotinib, ponatinib, dovitinib, rebastinib, CEP-701, ARRY-470, RXDX-101, LOXO-101, TSR-011, and PLX7486. Examples of HSP90 inhibitors include, but are not limited to, geldanamycin, herbimycin, 17-AAG, PU24FCI, STA-9090, IPI-504, and AUY-922. Examples of inhibitors that target tyrosine kinase receptor downstream signalling cascade include, without limitation, elumetinib and MK2206.

In some embodiments, a level of the marker is determined and compared to a standard level or reference range. In some embodiments, the standard level or reference range is determined according to a statistical procedure for risk prediction.

In some embodiments, the presence of the marker may be determined by detecting the presence of a polynucleotide or a polypeptide. In some embodiments, the method may comprise detecting the presence of the polypeptide using a reagent that specifically binds to the polypeptide or a fragment thereof. The reagent may be an antibody, an antibody derivative, or an antibody fragment.

In some embodiments, the presence of the marker may be determined by obtaining RNA from the sample; generating cDNA from the RNA; amplifying the cDNA with primers specific for the marker; and determining from the sequence of the amplified cDNA the presence of absence of the marker in the sample. In some embodiments, the presence of the marker may be determined by Fluorescent In Situ Hybridization (FISH).

The methods of the present invention may further comprise comparing the expression level of the marker in the sample to a control level of the marker selected from the group consisting of: a) a control level of the marker that has been correlated with beneficial response to the administration of a chemotherapeutic regimen including one or more kinase inhibitor(s); and a control level of the marker that has been correlated with lack of beneficial response to the administration of a chemotherapeutic regimen including one or more kinase inhibitor(s); and b) selecting the patient as being predicted to respond to the administration of a chemotherapeutic regimen including one or more kinase inhibitor(s), if the expression level of the marker in the sample is statistically similar to, or greater than, the control level of expression of the marker that has been correlated with sensitivity to the administration of a chemotherapeutic regimen including one or more kinase inhibitor(s), or c) selecting the patient as being predicted to not respond to the administration of a chemotherapeutic regimen including one or more kinase inhibitor(s), if the level of the marker in the sample is statistically less than the control level of the marker that has been correlated with beneficial response to the administration of a chemotherapeutic regimen including one or more kinase inhibitor(s).

In some embodiments, the methods may further comprise comparing the expression level of the marker in the sample to a level of the marker in a second patient predicted to not respond to the administration of a chemotherapeutic regimen including one or more kinase inhibitor(s), and, selecting the patient as being predicted to respond to the administration of a chemotherapeutic regimen including one or more kinase inhibitor(s), if the expression level of the marker in the sample is greater than the level of expression of the marker in the second patient, or, selecting the patient as being predicted to not respond to the administration of a chemotherapeutic regimen including one or more kinase inhibitor(s), if the level of the marker in the sample is less than or equal to the level of expression of the marker in the second patient. In some embodiments the patient is human.

In a further embodiment, the present invention includes an assay system for predicting patient response or outcome to tyrosine kinase anti-cancer therapy comprising a means to detect at least one of: a) the presence of a gene fusion selected from at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1; b) the level of expression of a gene transcript encoded by a gene fusion selected from at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1; c) the presence of a protein encoded by at least one gene fusion selected from at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1; d) the level of a protein encoded by at least a one gene fusion selected from at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1; and, e) the activity of a protein encoded by at least one gene fusion selected from at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1.

In some embodiments, the means to detect comprises nucleic acid probes comprising at least 10 to 50 contiguous nucleic acids of NTRK1 gene, or complementary nucleic acid sequences thereof. In some embodiments, the means to detect comprises binding ligands that specifically detect polypeptides encoded by selected from at least one gene fusion selected from C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1. In some embodiments, the assay system comprises a chip, array, or fluidity card. In some embodiments, the assay system further comprises: a control selected from the group consisting of: information containing a predetermined control level of a gene transcript encoded by at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and/or TCEANC2-NTRK1 gene fusion that has been correlated with response to the administration of a chemotherapeutic regimen including one or more kinase inhibitor(s); and information containing a predetermined control level of a gene transcript encoded by a at least one of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and/or TCEANC2-NTRK1 gene fusion that has been correlated with a lack of response to the administration of a chemotherapeutic regimen including one or more kinase inhibitor(s).

In another embodiment, the present invention includes a method of diagnosing a cancer in a subject, comprising detecting in a sample of cells from the subject the presence of a C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and/or TCEANC2-NTRK1 gene fusion marker, wherein the presence of the marker is indicative of whether the subject has cancer. In some embodiments, the presence of the gene fusion marker is detected by RT-PCR or FISH. In some embodiments, the presence of the gene fusion marker is detected by detecting the polypeptide encoded by the gene fusion marker. In some embodiments, the polypeptide is detected by using a reagent that specifically binds to the polypeptide or a fragment thereof.

Another embodiment is the use of a kit in the methods of this disclosure of identifying cancer patients that will respond to a tyrosine kinase inhibitor, wherein the kit comprises one or more of: an antibody, wherein the antibody specifically binds with a polypeptide marker, a labelled binding partner to the antibody, a solid phase upon which is immobilized the antibody or its binding partner, a polynucleotide probe that can hybridize to a polynucleotide marker, pairs of primers that under appropriate reaction conditions can prime amplification of at least a portion of a gene fusion polynucleotide marker (e.g., by PCR), instructions on how to use the kit, and a label or insert indicating regulatory approval for diagnostic or therapeutic use.

Another embodiment provides a tyrosine inhibitor drug for use in the treatment of a patient who is predicted to respond to the administration of a chemotherapeutic regimen in accordance with the methods of this disclosure of identifying cancer patients that will respond to a tyrosine kinase inhibitor.

In any of these methods of the invention, the cancer tissue sample may be obtained from a cancer tissue selected from a lung cancer, a liver cancer, a colorectal cancer, a thyroid cancer, a melanoma, a glioblastoma, and a glioma.

Other features and advantages of the invention will become apparent to one of skill in the art from the following detailed description.

### BRIEF DESCRIPTION OF THE DRAWINGS

The figures show the design and testing of the NTRK1 Break-Apart Probe. FIG. 1A shows the alignment of probes with genomic sequence from NTRK1 (REFSeq) showing 5′ (light lines) and 3′ (dark lines) probes. FIG. 1B shows the hybridization of the NTRK1 Break-Apart Probe with (Left panel) normal karyotype cells (GM09948) showing paired signals on chrom. 1, (middle panel) KM12 cells (colorectal cell line, NCI-60) demonstrating split 5′ and 3′ signals, and (right panel) FFPE tumor sample from patient with MPRIP-NTRK1 gene fusion demonstrating split 5′ and 3′ signals.

FIG. 2 shows FISH results and signal patterns from representative TMA samples.

FIG. 3 shows a schematic of the NTRK1 genomic region showing the placement of NTRK1-specific primers.

FIG. 4A shows a schematic for TRK-SHC1 PLA demonstrating detection of activated TRK. FIG. 4B shows TRK-SHC1 PLA applied to cell lines or FFPE tissue.

### DETAILED DESCRIPTION OF THE DISCLOSURE

The present inventors have discovered certain gene fusion events occurring with the NTRK1 gene that are indicative of the presence cancer and also indicative of cancer patient clinical response to treatment with tyrosine kinase inhibitors. These gene fusions, and the levels of the protein encoded by this gene fusion, along with clinical parameters can be used as biological markers to diagnose a cancer and to assess cancer patient response to treatment with tyrosine kinase inhibitors.

According to one definition, a biological marker is “a characteristic that is objectively measured and evaluated as an indicator of normal biologic processes, pathogenic processes, or pharmacological responses to therapeutic interventions” (NIH Marker Definitions Working Group (1998)). Biological markers can also include patterns or ensembles of characteristics indicative of particular biological processes (“panel of markers”). The marker measurement can be increased or decreased to indicate a particular biological event or process. In addition, if a marker measurement typically changes in the absence of a particular biological process, a constant measurement can indicate occurrence of that process.

Marker measurements may be of the absolute values (e.g., the molar concentration of a molecule in a biological sample) or relative values (e.g., the relative concentration of two molecules in a biological sample). The quotient or product of two or more measurements also may be used as a marker. For example, some physicians use the total blood cholesterol as a marker of the risk of developing coronary artery disease, while others use the ratio of total cholesterol to HDL cholesterol.

In the present invention, the markers may be used for diagnostic, prognostic, therapeutic, drug screening and patient stratification purposes (e.g., to group patients into a number of “subsets” for evaluation), as well as other purposes described herein, including evaluation of the effectiveness of a potential cancer therapeutic.

The terminology used herein is for describing particular embodiments and is not intended to be limiting. As used herein, the singular forms “a,” “and” and “the” include plural referents unless the content and context clearly dictate otherwise. Thus, for example, a reference to “a marker” includes a combination of two or more such markers. Unless defined otherwise, all scientific and technical terms are to be understood as having the same meaning as commonly used in the art to which they pertain. For the purposes of the present invention, the following terms are defined below.

As used herein, the term “marker” includes polypeptide markers and polynucleotide markers. For clarity of disclosure, aspects of the invention will be described with respect to “polypeptide markers” and “polynucleotide markers.” However, statements made herein with respect to “polypeptide markers” are intended to apply to other polypeptides of the invention. Likewise, statements made herein with respect to “polynucleotide” markers are intended to apply to other polynucleotides of the invention, respectively. Thus, for example, a polynucleotide described as encoding a “polypeptide marker” is intended to include a polynucleotide that encodes: a polypeptide marker, a polypeptide that has substantial sequence identity to a polypeptide marker, modified polypeptide markers, fragments of a polypeptide marker, precursors of a polypeptide marker and successors of a polypeptide marker, and molecules that comprise a polypeptide marker, homologous polypeptide, a modified polypeptide marker or a fragment, precursor or successor of a polypeptide marker (e.g., a fusion protein).

As used herein, the term “polypeptide” refers to a polymer of amino acid residues that has at least 5 contiguous amino acid residues, e.g., 5, 6, 7, 8, 9, 10, 11 or 12 or more amino acids long, including each integer up to the full length of the polypeptide. A polypeptide may be composed of two or more polypeptide chains. A polypeptide includes a protein, a peptide, an oligopeptide, and an amino acid. A polypeptide can be linear or branched. A polypeptide can comprise modified amino acid residues, amino acid analogs or non-naturally occurring amino acid residues and can be interrupted by non-amino acid residues. Included within the definition are amino acid polymers that have been modified, whether naturally or by intervention, e.g., formation of a disulfide bond, glycosylation, lipidation, methylation, acetylation, phosphorylation, or by manipulation, such as conjugation with a labeling component. Also included are antibodies produced by a subject in response to overexpressed polypeptide markers.

As used herein, a “fragment” of a polypeptide refers to a single amino acid or a plurality of amino acid residues comprising an amino acid sequence that has at least 5 contiguous amino acid residues, at least 10 contiguous amino acid residues, at least 20 contiguous amino acid residues or at least 30 contiguous amino acid residues of a sequence of the polypeptide. As used herein, a “fragment” of polynucleotide refers to a single nucleic acid or to a polymer of nucleic acid residues comprising a nucleic acid sequence that has at least 15 contiguous nucleic acid residues, at least 30 contiguous nucleic acid residues, at least 60 contiguous nucleic acid residues, or at least 90% of a sequence of the polynucleotide. In some embodiment, the fragment is an antigenic fragment, and the size of the fragment will depend upon factors such as whether the epitope recognized by an antibody is a linear epitope or a conformational epitope. Thus, some antigenic fragments will consist of longer segments while others will consist of shorter segments, (e.g. 5, 6, 7, 8, 9, 10, 11 or 12 or more amino acids long, including each integer up to the full length of the polypeptide). Those skilled in the art are well versed in methods for selecting antigenic fragments of proteins.

In some embodiments, a polypeptide marker is a member of a biological pathway. As used herein, the term “precursor” or “successor” refers to molecules that precede or follow the polypeptide marker or polynucleotide marker in the biological pathway. Thus, once a polypeptide marker or polynucleotide marker is identified as a member of one or more biological pathways, the present invention can include additional precursor or successor members of the biological pathway. Such identification of biological pathways and their members is within the skill of one in the art.

As used herein, the term “polynucleotide” refers to a single nucleotide or a polymer of nucleic acid residues of any length. The polynucleotide may contain deoxyribonucleotides, ribonucleotides, and/or their analogs and may be double-stranded or single stranded. A polynucleotide can comprise modified nucleic acids (e.g., methylated), nucleic acid analogs or non-naturally occurring nucleic acids and can be interrupted by non-nucleic acid residues. For example a polynucleotide includes a gene, a gene fragment, cDNA, isolated DNA, mRNA, tRNA, rRNA, isolated RNA of any sequence, recombinant polynucleotides, primers, probes, plasmids, and vectors. Included within the definition are nucleic acid polymers modified either naturally, or by intervention.

As used herein, a component (e.g., a marker) is referred to as “differentially expressed” in one sample as compared to another sample when the method used for detecting the component provides a different level or activity when applied to the two samples. A component is referred to as “increased” in the first sample if the method for detecting the component indicates that the level or activity of the component is higher in the first sample than in the second sample (or if the component is detectable in the first sample but not in the second sample). Conversely, a component is referred to as “decreased” in the first sample if the method for detecting the component indicates that the level or activity of the component is lower in the first sample than in the second sample (or if the component is detectable in the second sample but not in the first sample). In particular, marker is referred to as “increased” or “decreased” in a sample (or set of samples) obtained from a lung cancer subject (or a subject who is suspected of having lung cancer, or is at risk of developing lung cancer) if the level or activity of the marker is higher or lower, respectively, compared to the level of the marker in a sample (or set of samples) obtained from a non-lung cancer subject, or a reference value or range.

In order to determine the true frequency of NTRK1 fusions in non small cell lung cancer (NSCLC), the inventors performed fluorescence in situ hybridization (FISH) analysis using a NTRK1 break apart probe in a tumor microarray (TMA) of 447 surgically resected NSCLC patients. To confirm FISH results and to potentially identify novel NTRK1 fusions, selected NTRK1 FISH cases were also subjected to targeted next generation sequencing. The inventors discovered a host of previously unknown NTRK1 gene fusions indicative of cellular transformation that may be associated with constitutive activation of the kinase domain and thus its oncogenic capacity. These NTRK1 gene fusions include C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1.

The neurotrophic tyrosine kinase, receptor, type 1 (NTRK1) gene encodes the TrkA receptor tyrosine kinase. The NTRK1 gene has been isolated from a number of species such as human, chimpanzee, dog, cow, mouse, rat, chicken and zebrafish and the sequence determined. All these gene sequences are known to one skilled in the art and are intended to be encompassed in the present invention.

Chromosome 18 Open Reading Frame 8 (C18ORF8; NCBI GenBank: NC_000018.9, NT_010966.15, NC_018929.2) is a protein-coding gene associated with colon cancer. C18ORF8 encodes colon cancer-associated protein Mic1, which is a lysosomal membrane protein.

Ring Finger Protein 213 (RNF213; NC_000017.11) encodes a protein containing a C3HC4-type RING finger domain, which is a specialized type of Zn-finger that binds two atoms of zinc and is thought to be involved in mediating protein-protein interactions. The protein also contains an AAA domain, which is associated with ATPase activity. This gene is a susceptibility gene for Moyamoya disease, a vascular disorder of intracranial arteries. This gene is also a translocation partner in anaplastic large cell lymphoma and inflammatory myofibroblastic tumor cases, where a t(2;17)(p23;q25) translocation has been identified with the anaplastic lymphoma kinase (ALK) gene on chromosome 2, and a t(8;17)(q24;q25) translocation has been identified with the MYC gene on chromosome 8.

TBC1 domain family, member 22A (TBC1D22A; NC_000022.11) gene encodes the TBC1 domain family member 22A protein, which may play a role in obesity phenotype.

Nucleolar protein 4-like (C20ORF112 or NOL4L; NC_000020.11) gene has been implicated in translocations with AML and encodes a protein that may form stable associations with chromatin.

Delta/notch-like EGF repeat containing (DNER; NC_000002.12) gene has been associated with type 2 diabetes in American Indians and encodes the delta and Notch-like epidermal growth factor-related receptor protein.

NEL-like 1 (NELL1, NC_000011.10) gene encodes a cytoplasmic protein that contains epidermal growth factor (EGF)-like repeats. The encoded heterotrimeric protein may be involved in cell growth regulation and differentiation. Alternative splicing results in multiple transcript variants. The protein kinase C-binding protein NELL1 has been associated with Chagas cardiomyopathy, adverse metabolic response to HCTZ, lamotrigine- and phenytoin-induced hypersensitivity, the pathophysiology of childhood obesity, and as a novel IBD disease gene.

EPH-related receptor tyrosine kinase ligand 4 (EPL4; NC_000069.6) gene encodes the ephrin-A4 protein and appears to play a role in the developing visual system and has been associated with non-syndromic coronal synostosis.

Catenin (cadherin-associated protein), delta 2 (CTNND2; NC_000005.10) gene encodes an adhesive junction associated protein of the armadillo/beta-catenin superfamily and is implicated in brain and eye development and cancer formation. The catenin delta-2 protein encoded by this gene promotes the disruption of E-cadherin based adherens junction to favor cell spreading upon stimulation by hepatocyte growth factor. This gene is overexpressed in prostate adenocarcinomas and is associated with decreased expression of tumor suppressor E-cadherin in this tissue. This gene resides in a region of the short arm of chromosome 5 that is deleted in Cri du Chat syndrome. Alternative splicing results in multiple transcript variants encoding different isoforms.

Transcription elongation factor A (SII) N-terminal and central domain containing (2TCEANC2; NC_000001.11) encodes the transcription elongation factor A N-terminal and central domain-containing protein 2 protein.

A fluorescence in situ hybridization (FISH) assay was used to detect chromosomal rearrangements within the NTRK1 gene in formalin-fixed, paraffin-embedded (FFPE) tissue sections. FISH probes that detect other NTRK1 gene fusions, regardless of the specific 5′ gene fusion partner, were also used. (See Examples 2 and 4.)

The dual-color FISH assay is conducted using the inventors' NTRK1 break-apart probe (3′ NTRK1 [SpectrumRed] and 5′ NTRK1 [SpectrumGreen]). Pre-hybridization treatments was performed using the reagents from the Vysis Paraffin Kit IV (Abbott Molecular). Samples were deemed positive for NTRK1 rearrangement if ≧15% of tumor cells demonstrated an isolated 3′ signal or a separation of 5′ and 3′ signals that was greater than one signal diameter.

The NTRK1 gene fusion biomarkers identified herein serve as a novel diagnostic markers of cancer and cancer patient response to certain anti-cancer drugs. NTRK1 gene encodes the TRKA receptor tyrosine kinase. Small molecule tyrosine kinase inhibitors inhibit activated TRKA. It is believed that the NTRK1 gene fusion markers identified herein are indicators of cancer patient response to tyrosine kinase inhibitors. Accordingly, in one aspect, the invention provides gene fusion biomarkers, the presence or expression level of which are indicative of cancer patient response to tyrosine kinase inhibitors.

In another aspect, the gene fusion markers of the present invention can serve as indicators of cancer patient response to other targeted cancer therapies such as administration of HSP90 inhibitors (or other chaperone inhibitors) or agents that target downstream signalling cascades. Such inhibitors are well known in the art and are commercially available. All such inhibitors are encompassed in the present invention. Examples of HSP90 inhibitors include without limitation geldanamycin, herbimycin, 17-AAG, PU24FCI, STA-9090, IPI-504, and AUY-922. Examples of agents that target downstream signalling cascades include selumetinib and MK2206.

The presence of the marker may be detected by detecting a polynucleotide. In one embodiment, the polynucleotide may be a probe that specifically hybridizes with the NTRK1 gene sequences and identifies a chromosomal rearrangement involving the NTRK1 gene. In another embodiment, the polynucleotide may be a primer that specifically binds and amplifies a polynucleotide sequence that is indicative of the presence of the gene fusion involving a NTRK1 gene, including at least one of the C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1 gene fusion markers.

Some variation is inherent in the measurements of the physical and chemical characteristics of the markers of the invention. The magnitude of the variation depends to some extent on the reproducibility of the separation means and the specificity and sensitivity of the detection means used to make the measurement. Preferably, the method and technique used to measure the markers is sensitive and reproducible.

The presence of the gene fusion marker may also be detected by detecting a polynucleotide. Polypeptides corresponding to the NTRK1 gene fusion markers may include a fragment, precursor, successor or modified version of the protein encoded by the NTRK1-gene fusion markers. In another embodiment, the invention includes a molecule that comprises a fragment, precursor, successor or modified polypeptide encoded by the NTRK1-gene fusion markers.

Another embodiment of the present invention relates to an assay system including a plurality of antibodies, or antigen binding fragments thereof, or aptamers for the detection of the expression of the NTRK1 gene fusion markers of the invention. The plurality of antibodies, or antigen binding fragments thereof, or aptamers selectively bind to proteins encoded by the NTRK1 gene fusion markers.

As used herein, the terms “patient,” “subject,” “a subject who has cancer” and “cancer patient” are intended to refer to subjects who have been diagnosed with a cancer or are suspected of having cancer. The terms “non-subject” and “a subject who does not have cancer” are intended to refer to a subject who has not been diagnosed with cancer, or who is cancer-free as a result of surgery to remove one or more tumors. A non-cancer subject may be healthy and have no other disease, or they may have a disease other than cancer. The NTRK1 gene has been found to be conserved in a number of species such as chimpanzee, dog, cow, mouse, rat, chicken, and zebrafish and their sequences are known. In some embodiments, the patient or subject may be a mammal. In a preferred embodiment, the patient or subject is human.

Polypeptides encoded by the NTRK1 gene fusion may be isolated by any suitable method known in the art. Native polypeptides encoded by the NTRK1 gene fusion can be purified from natural sources by standard methods known in the art (e.g., chromatography, centrifugation, differential solubility, immunoassay). In one embodiment, the polypeptides may be isolated from a tumor sample. In another embodiment, the polypeptides may be isolated from a sample by contacting the sample with substrate-bound antibodies or aptamers that specifically bind to the marker.

The present invention also includes polynucleotides related to the gene fusion markers of the present invention. In one aspect, the invention provides polynucleotides that comprise the NTRK1 gene fusion markers of the invention. These may be referred to as polynucleotide markers. The polynucleotide markers may be genomic DNA, cDNA, or mRNA transcripts. In another embodiment, the invention provides polynucleotides that have substantial sequence similarity to a polynucleotide that comprises the NTRK1 gene fusion markers or variants thereof, including the NTRK1-gene fusion markers.

In some embodiments, the polypeptides encoded by the NTRK1 gene fusion markers i.e. polypeptide markers may be used as surrogate markers of the NTRK1 gene fusions of this disclosure. Thus, for example, if a polypeptide encoded by the NTRK1 gene fusion markers of this invention are present in cancer patients, the presence or level or activity of the polypeptides may be interrogated (e.g., to identify cancer patients expected to respond to tyrosine kinase inhibitors).

Polynucleotide markers comprising the gene fusion markers may be isolated by any suitable method known in the art. Native polynucleotide markers may be purified from natural sources by standard methods known in the art (e.g., chromatography, centrifugation, differential solubility, immunoassay). In one embodiment, a polynucleotide marker may be isolated from a mixture by contacting the mixture with substrate bound probes that are complementary to the polynucleotide marker under hybridization conditions.

Alternatively, polynucleotide markers comprising the NTRK1 gene fusion may be synthesized by any suitable chemical or recombinant method known in the art. In one embodiment, for example, the makers can be synthesized using the methods and techniques of organic chemistry. In another embodiment, a polynucleotide marker can be produced by polymerase chain reaction (PCR).

The present invention also encompasses molecules which specifically bind the polypeptide or polynucleotide markers of the present invention. In one aspect, the invention provides molecules that specifically bind to a polypeptide marker or a polynucleotide marker. As used herein, the term “specifically binding,” refers to the interaction between binding pairs (e.g., an antibody and an antigen or aptamer and its target). In some embodiments, the interaction has an affinity constant of at most 10⁻⁶ moles/liter, at most 10⁻⁷ moles/liter, or at most 10⁻⁸ moles/liter. In other embodiments, the phrase “specifically binds” refers to the specific binding of one protein to another (e.g., an antibody, fragment thereof, or binding partner to an antigen), wherein the level of binding, as measured by any standard assay (e.g., an immunoassay), is statistically significantly higher than the background control for the assay. For example, when performing an immunoassay, controls typically include a reaction well/tube that contain antibody or antigen binding fragment alone (i.e., in the absence of antigen), wherein an amount of reactivity (e.g., non-specific binding to the well) by the antibody or antigen binding fragment thereof in the absence of the antigen is considered to be background. Binding can be measured using a variety of methods standard in the art including enzyme immunoassays (e.g., ELISA), immunoblot assays, etc.).

The binding molecules include antibodies, aptamers and antibody fragments. As used herein, the term “antibody” refers to an immunoglobulin molecule capable of binding an epitope present on an antigen. The term is intended to encompasses not only intact immunoglobulin molecules such as monoclonal and polyclonal antibodies, but also bi-specific antibodies, humanized antibodies, chimeric antibodies, anti-idiopathic (anti-ID) antibodies, single-chain antibodies, Fab fragments, F(ab′) fragments, fusion proteins and any modifications of the foregoing that comprise an antigen recognition site of the required specificity. As used herein, an aptamer is a non-naturally occurring nucleic acid having a desirable action on a target. A desirable action includes, but is not limited to, binding of the target, catalytically changing the target, reacting with the target in a way which modifies/alters the target or the functional activity of the target, covalently attaching to the target as in a suicide inhibitor, facilitating the reaction between the target and another molecule. In a preferred embodiment, the action is specific binding affinity for a target molecule, such target molecule being a three dimensional chemical structure other than a polynucleotide that binds to the nucleic acid ligand through a mechanism which predominantly depends on Watson/Crick base pairing or triple helix binding, wherein the nucleic acid ligand is not a nucleic acid having the known physiological function of being bound by the target molecule.

Certain antibodies that specifically bind polypeptide markers polynucleotide markers of the invention already may be known and/or available for purchase from commercial sources. In any event, the antibodies of the invention may be prepared by any suitable means known in the art. For example, antibodies may be prepared by immunizing an animal host with a marker or an immunogenic fragment thereof (conjugated to a carrier, if necessary). Adjuvants (e.g., Freund's adjuvant) optionally may be used to increase the immunological response. Sera containing polyclonal antibodies with high affinity for the antigenic determinant can then be isolated from the immunized animal and purified.

Alternatively, antibody-producing tissue from the immunized host can be harvested and a cellular homogenate prepared from the organ can be fused to cultured cancer cells. Hybrid cells which produce monoclonal antibodies specific for a marker can be selected. Alternatively, the antibodies of the invention can be produced by chemical synthesis or by recombinant expression. For example, a polynucleotide that encodes the antibody can be used to construct an expression vector for the production of the antibody. The antibodies of the present invention can also be generated using various phage display methods known in the art.

Antibodies or aptamers that specifically bind markers of the invention can be used, for example, in methods for detecting protein products encoded by the NTRK1 gene fusion markers of the invention. In one embodiment, antibodies or aptamers against a polypeptide marker or polynucleotide marker of the invention can be used to assay a tissue sample (e.g., a thin cortical slice) for the markers. The antibodies or aptamers can specifically bind to the marker, if any, present in the tissue sections and allow the localization of the marker in the tissue. Similarly, antibodies or aptamers labelled with a radioisotope may be used for in vivo imaging or treatment applications.

The present invention also provides methods of detecting the NTRK1 gene fusion markers of the present invention. The markers of the invention may be detected by any method known to those of skill in the art, including without limitation LC-MS, GC-MS, immunoassays, hybridization and enzyme assays. The detection may be quantitative or qualitative. A wide variety of conventional techniques are available, including mass spectrometry, chromatographic separations, 2-D gel separations, binding assays (e.g., immunoassays), competitive inhibition assays, and so on. Any effective method in the art for measuring the presence/absence, level or activity of a polypeptide or polynucleotide is included in the invention. It is within the ability of one of ordinary skill in the art to determine which method would be most appropriate for measuring a specific marker. Thus, for example, an ELISA assay may be best suited for use in a physician's office while a measurement requiring more sophisticated instrumentation may be best suited for use in a clinical laboratory. Regardless of the method selected, it is important that the measurements be reproducible.

For protein markers, quantification can be based on derivatization in combination with isotopic labelling, referred to as isotope coded affinity tags (“ICAT”). In this and other related methods, a specific amino acid in two samples is differentially and isotopically labelled and subsequently separated from peptide background by solid phase capture, wash and release. The intensities of the molecules from the two sources with different isotopic labels can then be accurately quantified with respect to one another. Quantification can also be based on the isotope dilution method by spiking in an isotopically labelled peptide or protein analogous to those being measured. Furthermore, quantification can also be determined without isotopic standards using the direct intensity of the analyte comparing with another measurement of a standard in a similar matrix.

In addition, one- and two-dimensional gels have been used to separate proteins and quantify gels spots by silver staining, fluorescence or radioactive labelling. These differently stained spots have been detected using mass spectrometry, and identified by tandem mass spectrometry techniques.

A number of the assays discussed above employ a reagent that specifically binds to a NTRK1 gene fusion marker of the invention. Any molecule that is capable of specifically binding to the NTRK1 gene fusion markers of the invention is included within the invention. In some embodiments, the binding molecules are antibodies or antibody fragments. In other embodiments, the binding molecules are non-antibody species, such as aptamers or nucleotide probes. As described above, the binding molecules may be identified and produced by any method accepted in the art. Methods for identifying and producing antibodies and antibody fragments specific for an analyte are well known.

The markers of the invention also may be detected or measured using a number of chemical derivatization or reaction techniques known in the art. Reagents for use in such techniques are known in the art, and are commercially available for certain classes of target molecules.

Measurement of the relative amount of an RNA or protein marker of the invention may be by any method known in the art (see, e.g., Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989; and Current Protocols in Molecular Biology, eds. Ausubel et al. John Wiley & Sons: 1992). Typical methodologies for RNA detection include RNA extraction from a cell or tissue sample, followed by hybridization of a labelled probe (e.g., a complementary polynucleotide) specific for the target RNA to the extracted RNA, and detection of the probe (e.g., Northern blotting). Typical methodologies for protein detection include protein extraction from a cell or tissue sample, followed by hybridization of a labelled probe (e.g., an antibody) specific for the target protein to the protein sample, and detection of the probe. The label group can be a radioisotope, a fluorescent compound, an enzyme, or an enzyme co-factor. Detection of specific protein and polynucleotides may also be assessed by gel electrophoresis, column chromatography, direct sequencing, or quantitative PCR (in the case of polynucleotides) among many other techniques well known to those skilled in the art.

Detection of the presence or number of copies of all or a part of a marker gene of the invention may be performed using any method known in the art. Typically, it is convenient to assess the presence and/or quantity of a DNA or cDNA by Southern analysis, in which total DNA from a cell or tissue sample is extracted, is hybridized with a labelled probe (e.g., a complementary DNA molecule), and the probe is detected. The label group can be a radioisotope, a fluorescent compound, an enzyme, or an enzyme co-factor. Other useful methods of DNA detection and/or quantification include direct sequencing, gel electrophoresis, column chromatography, and quantitative PCR, as is known by one skilled in the art.

Polynucleotide similarity can be evaluated by hybridization between single stranded nucleic acids with complementary or partially complementary sequences. Such experiments are well known in the art. High stringency hybridization and washing conditions, as referred to herein, refer to conditions which permit isolation of nucleic acid molecules having at least about 80% nucleic acid sequence identity with the nucleic acid molecule being used to probe in the hybridization reaction (i.e., conditions permitting about 20% or less mismatch of nucleotides). Very high stringency hybridization and washing conditions, as referred to herein, refer to conditions which permit isolation of nucleic acid molecules having at least about 90% nucleic acid sequence identity with the nucleic acid molecule being used to probe in the hybridization reaction (i.e., conditions permitting about 10% or less mismatch of nucleotides). One of skill in the art can calculate the appropriate hybridization and wash conditions to achieve these particular levels of nucleotide mismatch. Such conditions will vary, depending on whether DNA:RNA or DNA:DNA hybrids are being formed. Calculated melting temperatures for DNA:DNA hybrids are 10° C. less than for DNA:RNA hybrids. In particular embodiments, stringent hybridization conditions for DNA:DNA hybrids include hybridization at an ionic strength of 6×SSC (0.9 M Na⁺) at a temperature of between about 20° C. and about 35° C. (lower stringency), more preferably, between about 28° C. and about 40° C. (more stringent), and even more preferably, between about 35° C. and about 45° C. (even more stringent), with appropriate wash conditions. In particular embodiments, stringent hybridization conditions for DNA:RNA hybrids include hybridization at an ionic strength of 6×SSC (0.9 M Na⁺) at a temperature of between about 30° C. and about 45° C., more preferably, between about 38° C. and about 50° C., and even more preferably, between about 45° C. and about 55° C., with similarly stringent wash conditions. These values are based on calculations of a melting temperature for molecules larger than about 100 nucleotides, 0% formamide and a G+C content of about 40%. Alternatively, Tₘ can be calculated empirically as set forth in Sambrook et al., supra, pages 9.31 to 9.62. In general, the wash conditions should be as stringent as possible, and should be appropriate for the chosen hybridization conditions. For example, hybridization conditions can include a combination of salt and temperature conditions that are approximately 20-25° C. below the calculated Tₘ of a particular hybrid, and wash conditions typically include a combination of salt and temperature conditions that are approximately 12-20° C. below the calculated Tₘ of the particular hybrid. One example of hybridization conditions suitable for use with DNA:DNA hybrids includes a 2-24 hour hybridization in 6×SSC (50% formamide) at about 42° C., followed by washing steps that include one or more washes at room temperature in about 2×SSC, followed by additional washes at higher temperatures and lower ionic strength (e.g., at least one wash as about 37° C. in about 0.1×-0.5×SSC, followed by at least one wash at about 68° C. in about 0.1×-0.5×SSC). Other hybridization conditions, and for example, those most useful with nucleic acid arrays, will be known to those of skill in the art.

Using the methods of the present invention, administration of a chemotherapeutic drug or drug combination can be evaluated or re-evaluated in light of the assay results of the present invention. For example, the tyrosine kinase inhibitor drug(s) can be administered differently to different subject populations, depending on the presence of the NTRK1-gene fusion markers of the invention in tumor samples from the subjects tested. Results from the different drug regimens can also be compared with each other directly. Alternatively, the assay results may indicate the desirability of one drug regimen over another, or indicate that a specific drug regimen should or should not be administered to a cancer patient. In one preferred embodiment, the finding of the presence of the NTRK1 gene fusion markers of the invention is indicative of a good prognosis for response to treatment with chemotherapeutic agents comprising tyrosine kinase inhibitors (“tyrosine kinase inhibitor chemotherapeutic agents”). In another preferred embodiment, the absence of the NTRK1 gene fusion markers of the invention in a cancer patient is indicative of a poor prognosis for response to treatment with tyrosine kinase inhibitor chemotherapeutic agents, and may further recommend not administering tyrosine kinase inhibitor chemotherapeutic agent drug regimens.

In another aspect, the invention provides a kit for identifying cancer patients predicted to respond or not respond to tyrosine kinase inhibitor drugs, based on the presence or absence of NTRK1 gene fusion markers of this disclosure.

The kits of the invention may comprise one or more of the following: an antibody, wherein the antibody specifically binds with a polypeptide marker, a labelled binding partner to the antibody, a solid phase upon which is immobilized the antibody or its binding partner, a polynucleotide probe that can hybridize to a polynucleotide marker, pairs of primers that under appropriate reaction conditions can prime amplification of at least a portion of a gene fusion polynucleotide marker (e.g., by PCR), instructions on how to use the kit, and a label or insert indicating regulatory approval for diagnostic or therapeutic use.

The invention further includes polynucleotide or polypeptide microarrays comprising polypeptides of the invention, polynucleotides of the invention, or molecules, such as antibodies, which specifically bind to the polypeptides or polynucleotides of the present invention. In this aspect of the invention, standard techniques of microarray technology are utilized to assess expression of the polypeptides markers and/or identify biological constituents that bind such polypeptides. Protein microarray technology is well known to those of ordinary skill in the art and is based on, but not limited to, obtaining an array of identified peptides or proteins on a fixed substrate, binding target molecules or biological constituents to the peptides, and evaluating such binding. Polynucleotide arrays, particularly arrays that bind polypeptides of the invention, also can be used for diagnostic applications, such as for identifying subjects that have a condition characterized by expression of polypeptide markers, e.g., cancer.

The assay systems of the present invention can include a means for detecting in a sample of tumor cells the presence of the NTRK1 gene fusion markers of the invention, and/or a level of expression of the NTRK1 gene fusion markers of the invention, and/or a level of protein product of the NTRK1 gene fusion markers of the invention.

The assay system preferably also includes one or more controls. The controls may include: (i) a control sample for detecting sensitivity to tyrosine kinase inhibitor chemotherapeutics; (ii) a control sample for detecting resistance to tyrosine kinase inhibitor chemotherapeutics; (iii) information containing a predetermined control level of markers to be measured with regard to tyrosine kinase inhibitor sensitivity or resistance (e.g., a predetermined control level of a marker of the NTRK1 gene fusion of the present invention that has been correlated with sensitivity to tyrosine kinase inhibitor chemotherapeutics or resistance to tyrosine kinase inhibitor chemotherapeutics).

In another embodiment, a means for detecting the NTRK1 gene fusion markers of the disclosure can generally be any type of reagent that can include, but are not limited to, polynucleotides, hybridization probes, PCR primers, antibodies and antigen binding fragments thereof, peptides, binding partners, aptamers, enzymes, and small molecules. Additional reagents useful for performing an assay using such means for detection can also be included, such as reagents for performing immunohistochemistry, Fluorescent in situ Hybridization (FISH) or a preferred binding assay.

The means for detecting of the assay system of the present invention can be conjugated to a detectable tag or detectable label. Such a tag can be any suitable tag which allows for detection of the reagents used to detect the gene or protein of interest and includes, but is not limited to, any composition or label detectable by spectroscopic, photochemical, electrical, optical or chemical means. Useful labels in the present invention include: biotin for staining with labeled streptavidin conjugate, magnetic beads (e.g., DYNABEADS™), fluorescent dyes (e.g., fluorescein, texas red, rhodamine, green fluorescent protein, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold or colored glass or plastic (e.g., polystyrene, polypropylene, latex, etc.) beads.

In addition, the means for detecting of the assay system of the present invention can be immobilized on a substrate. Such a substrate can include any suitable substrate for immobilization of a detection reagent such as would be used in any of the previously described methods of detection. Briefly, a substrate suitable for immobilization of a means for detecting includes any solid support, such as any solid organic, biopolymer or inorganic support that can form a bond with the means for detecting without significantly affecting the activity and/or ability of the detection means to detect the desired target molecule. Exemplary organic solid supports include polymers such as polystyrene, nylon, phenol-formaldehyde resins, and acrylic copolymers (e.g., polyacrylamide). The kit can also include suitable reagents for the detection of the reagent and/or for the labeling of positive or negative controls, wash solutions, dilution buffers and the like. The assay system can also include a set of written instructions for using the system and interpreting the results.

The assay system can also include a means for detecting a control marker that is characteristic of the cell type being sampled can generally be any type of reagent that can be used in a method of detecting the presence of a known marker (at the nucleic acid or protein level) in a sample, such as by a method for detecting the presence of a marker described previously herein. Specifically, the means is characterized in that it identifies a specific marker of the cell type being analyzed that positively identifies the cell type. For example, in a lung tumor assay, it is desirable to screen lung cancer cells for the level of the marker expression and/or biological activity. Therefore, the means for detecting a control marker identifies a marker that is characteristic of, for example, a lung cell, so that the cell is distinguished from other cell types, such as a connective tissue or inflammatory cell. Such a means increases the accuracy and specificity of the assay of the present invention. Such a means for detecting a control marker include, but are not limited to: a probe that hybridizes under stringent hybridization conditions to a nucleic acid molecule encoding a protein marker; PCR primers which amplify such a nucleic acid molecule; an aptamer that specifically binds to a conformationally-distinct site on the target molecule; and/or an antibody, antigen binding fragment thereof, or antigen binding peptide that selectively binds to the control marker in the sample. Nucleic acid and amino acid sequences for many cell markers are known in the art and can be used to produce such reagents for detection.

The assay systems and methods of the present invention can be used not only to identify patients that are predicted to be responsive to tyrosine kinase inhibitor chemotherapeutic agents, but also to identify treatments that can improve the responsiveness of cancer cells which are resistant to tyrosine kinase inhibitor chemotherapeutic agents, and to develop adjuvant treatments that enhance the response of cancer patients to tyrosine kinase inhibitor chemotherapeutic agent(s).

The Examples that follow are illustrative of specific embodiments of the invention, and various uses thereof. They are set forth for explanatory purposes only, and are not to be taken as limiting the invention.

### EXAMPLES

### Example 1: This Example Illustrates the FISH Assay Performed for Detecting the Presence of NTRK1 Gene Fusion Events

In order to determine the true frequency of NTRK1 fusions in NSCLC, the inventors performed fluorescence in situ hybridization (FISH) analysis using a NTRK1 break apart probe in a tumor microarray (TMA) of 447 surgically resected non small cell lung cancer (NSCLC) patients. The tumor microarray used in this study was created from 447 surgically resected patients that received a radical resection of a primary NSCLC during the period 2000-2004. The patient characteristics of the TMA are listed in Table 1.

TABLE 1
Characteristics of patients samples in TMA

| Characteristic | number | % |
|---|---|---|
| Total | 447 | |
| Median age, years | 66 | |
| Range | 33-86 | |
| Sex | | |
| Male | 373 | 83.4 |
| Female | 74 | 16.6 |
| Smoking history | | |
| Never | 40 | 8.9 |
| Current/former | 389 | 87 |
| Unknown | 18 | 4.1 |
| Histology | | |
| Adenocarcinoma | 244 | 54.6 |
| Squamous cell carcinoma | 138 | 30.9 |
| Neuroendocrine | 27 | 6 |
| Large cell | 6 | 1.3 |
| Undifferentiated | 32 | 7.2 |
| Pathologic stage | | |
| I | 166 | 37.1 |
| II | 99 | 22.1 |
| IIIa | 122 | 27.3 |
| IIIb | 24 | 5.4 |
| IV | 36 | 8.1 |

Three tissue cores (0.6 mm diameters) were available from each patient and a minimum of 30 tumor cells (range 30-90) were scored.

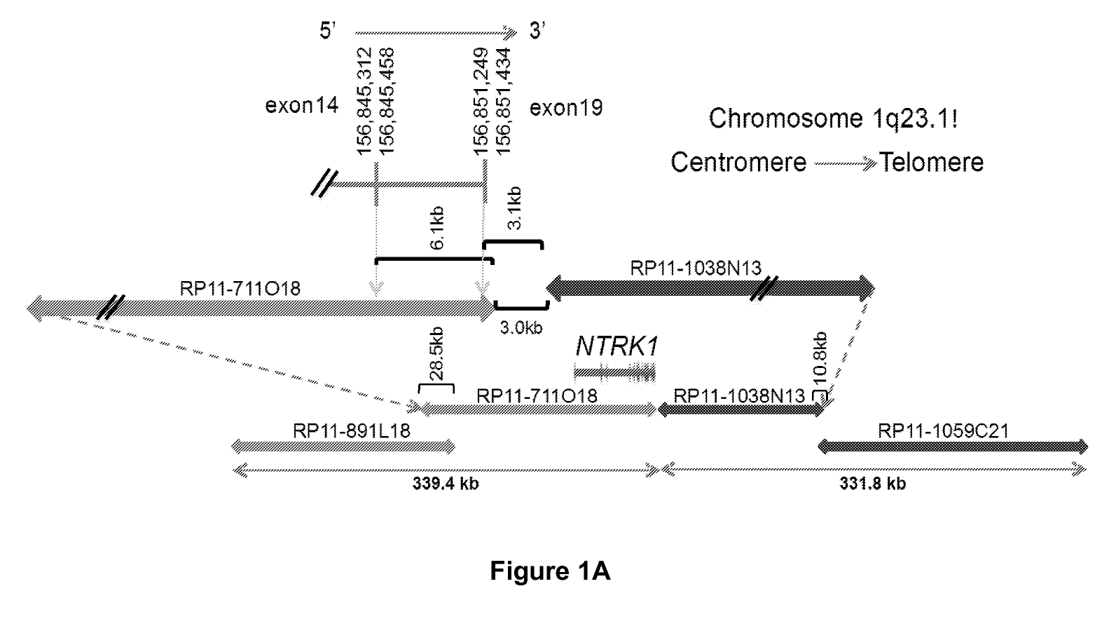

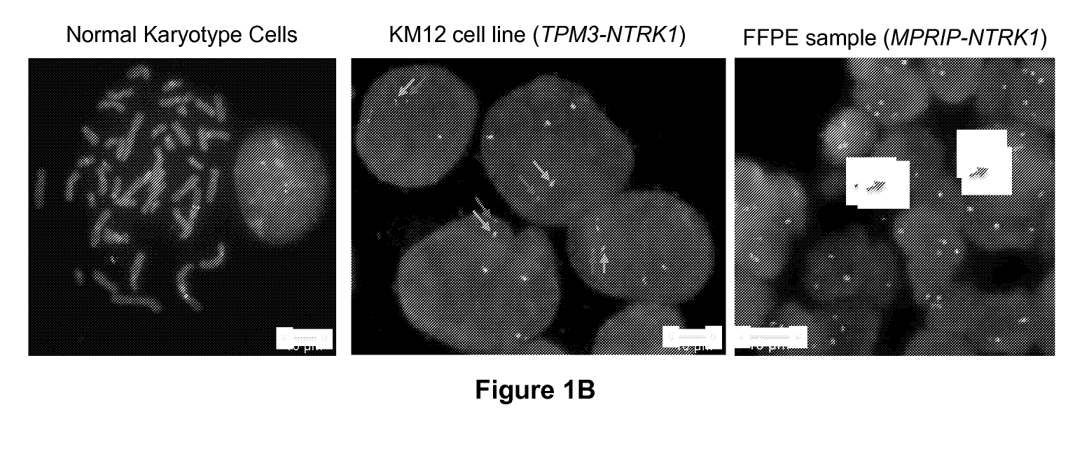

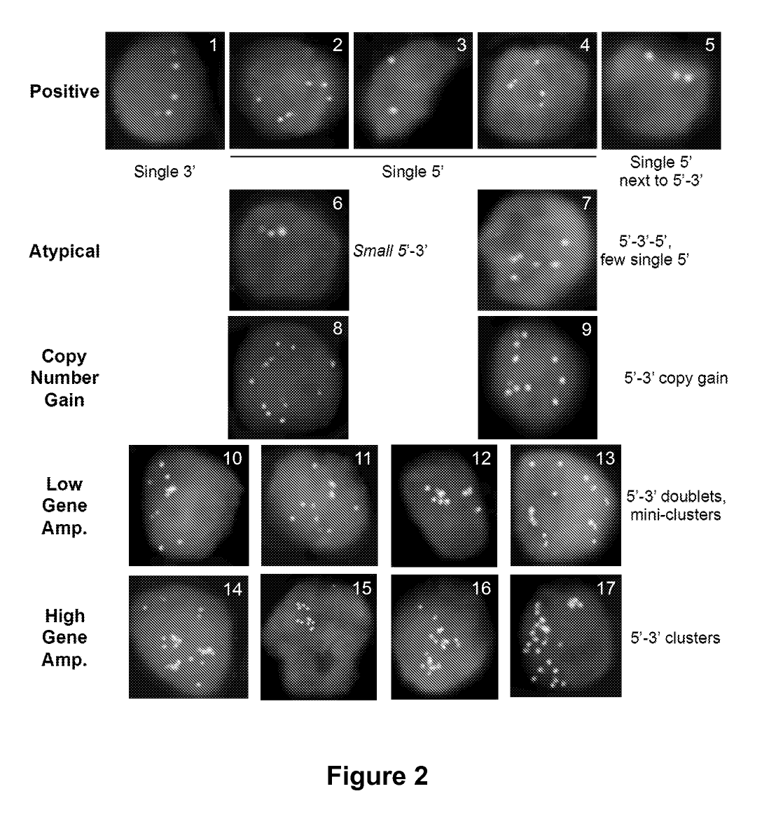

The analyses conducted using the NTRK1 break-apart probe were performed under a 100× immersion oil objective using fluorescence microscopes equipped with Texas Red, FITC and DAPI single band pass filters, dual and triple pass filters. Images were captured by CCD cameras using the CytoVision FISH software (Leica microsystems). Red (3′) and green (5′) signals physically separated by ≧2 signal diameters were considered split. A specimen was considered positive for NTRK1 rearrangement if ≧15% of the cells showed split signals, single 5′ signals, or single 3′ signals. Additional samples with high frequency of extra or atypical signals were also selected for further analysis. The atypical patterns indicated occurrence of chromosomal changes (mostly tandem duplications) that may mask cryptic gene fusions. The inventors identified 5/443 NSCLC tumors that were FISH positive, showing evidence of an NTRK1 gene rearrangement. Twelve additional tumor samples with atypical FISH patterns or gene amplification were identified that are indicative of oncogenic NTRK1 alterations. FIG. 2 shows FISH results and signal patterns from representative tumor nuclei from 17 TMA samples. Characteristics of the patients from the 17 representative tumor nuclei shown in FIG. 2 are provided in Table 2.

TABLE 2
Characteristics of Selected Patients with NTRK1 FISH Results

| | | | Smoking | | | ALK, ROS1, | |
|---|---|---|---|---|---|---|---|
| Patient # | Sex | Age | Status | Stage | Histology^{§} | RET, EGFR^{¶} | FISH Result |
| 1 | F | 70 | Current | 2 | NE | Negative* | Positive |
| 2 | M | 47 | Unknown | 3 | ADC | Negative* | Positive |
| 3 | M | 65 | Current | 2 | SCC | Negative* | Positive |
| 4 | F | 76 | Never | 4 | ADC | Negative* | Positive |
| 5 | F | 71 | Never | 1 | ADC | EGFR L858R | Positive |
| 6 | M | 75 | Current | 2 | ADC | Negative | Atypical |
| 7 | F | 66 | Never | 3 | ADC | Negative* | Atypical |
| 8 | M | 52 | Former | 1 | ADC | Negative | Copy Number Gain |
| 9 | M | 72 | Former | 4 | ADC | Negative* | Copy Number Gain |
| 10 | M | 81 | Former | 2 | ADC | Negative | Low Gene Amp. |
| 11 | M | 69 | Current | 1 | ADC | Negative | Low Gene Amp. |
| 12 | M | 70 | Current | 2 | ADC | Negative | Low Gene Amp. |
| 13 | M | 61 | Former | 1 | ADC | Negative | Low Gene Amp. |
| 14 | F | 56 | Former | 3 | ADC | Negative* | High Gene Amp. |
| 15 | M | 69 | Former | 3 | ADC | Negative* | High Gene Amp. |
| 16 | M | 49 | Former | 3 | ADC | Negative* | High Gene Amp. |
| 17 | M | 77 | Former | 1 | ADC | Negative | High Gene Amp. |

^{§}ADC = Adenocarcinoma, SCC = Squamous cell carcinoma, NE = neuroendocrine
^{¶}Oncogenes analyzed include ALK, ROS1, RET (FISH) and EGFR (HRM)
*EGFR mutation status not available

### Example 2: This Example Illustrates the Sequencing Analysis Performed to Identify Novel NTRK1 Gene Fusion Events

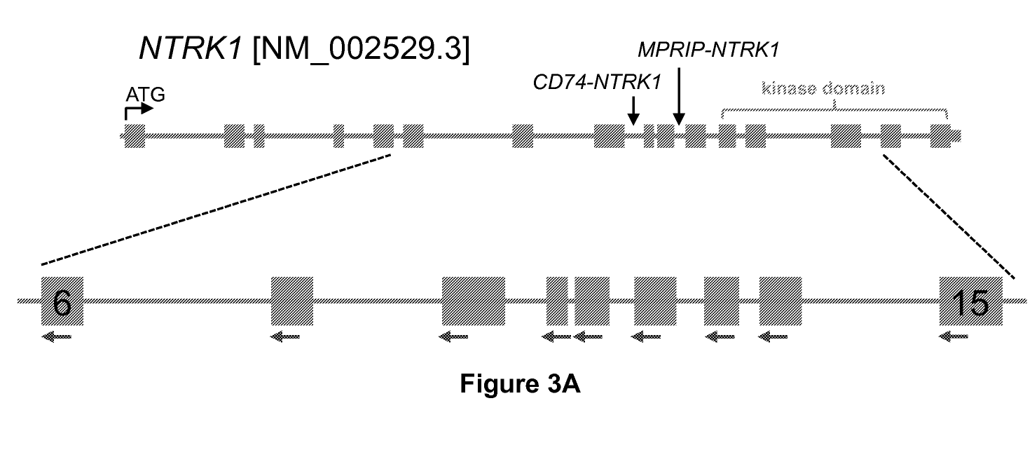

To confirm FISH results and to potentially identify novel NTRK1 fusions, selected NTRK1 FISH cases were also subjected to targeted next generation sequencing. Total nucleic acids were extracted from FFPE tumor blocks corresponding to the selected TMA cores using the Agencourt FormaPure Kit (Beckman Coulter). Library preparation was conducted with an ARCHER™ Custom Fusion Assay (Enzymatics) that included primers corresponding to exons 6, 7, 8, 10, 11, 12, 13, 14 and 15 of NTRK1 (NM_002529.3). Samples were run on a MiSeq Desktop Sequencer (Illumina) and data was analyzed using ARCHER™ Analysis Pipeline. FIG. 3 shows a schematic of the NTRK1 genomic region showing the placement of NTRK1-specific primers.

The confirmatory analysis by next generation sequencing (NGS) performed using NTRK1-anchored exon sequences on selected samples based on FISH patterns identified the following NTRK1 gene fusions, which are predicted to result in an in-frame transcript and contain an intact TRKA kinase domain:

C18ORF8-NTRK1

RNF213-NTRK1

TBC1 D22A-NTRK1

C20ORF112-NTRK1

DNER-NTRK1

NELL1-NTRK1

EPL4-NTRK1

CTNND2-NTRK1, and

TCEANC2-NTRK1.

### Example 3: This Example Illustrates the Proximity Ligation Assay (PLA) Used to Detect Activated TRK

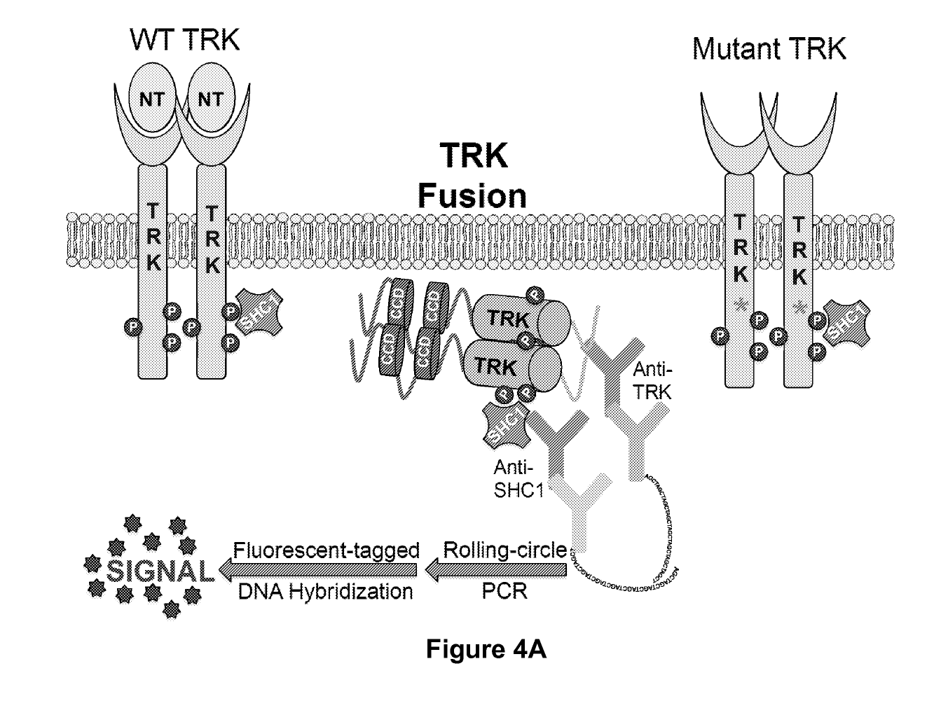

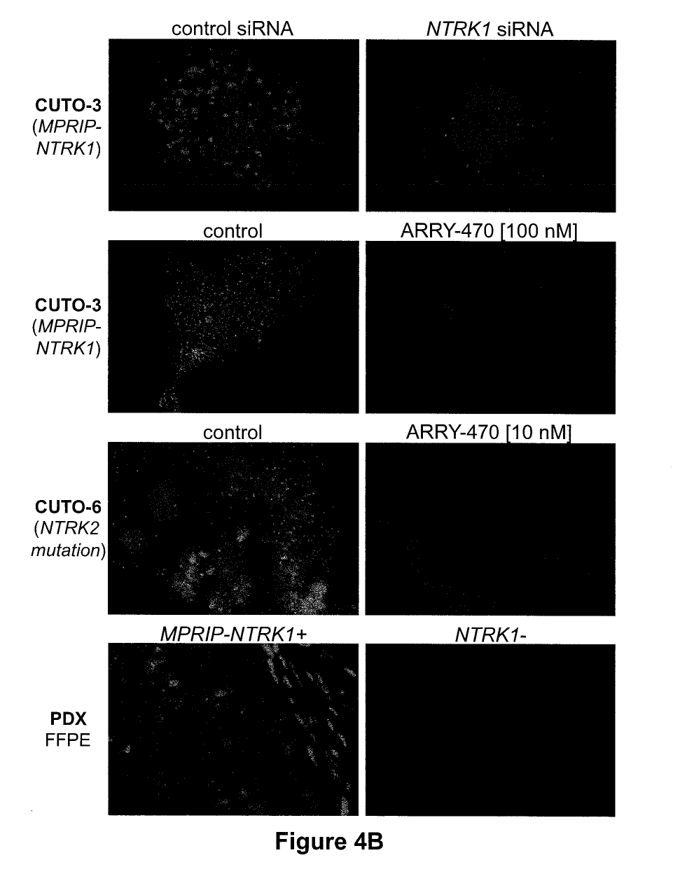

The inventors also demonstrated the use of a new method, a TRK-SHC1 proximity ligation assay (PLA), which detects activated TRK in tumor cells regardless of the mechanism of activation. FIG. 4A provides a schematic for TRK-SHC1 PLA demonstrating detection of activated TRK regardless of mechanism of activation (gene fusion fusion, mutation, or autocrine/paracrine activation of WT) or family member (TRKA/B/C). The Duolink Proximity Ligation Assay (Sigma Aldrich) was used according to manufactures instructions. To detect TRK-SHC1 complexes, anti-TRK (rabbit, pan-TRK antibody that recognizes TRKA, TRKB, and TRKC; Cell Signaling) and the anti-SHC1 (mouse; Novus Biologicals) were applied to cell lines grown on slides or FFPE tissue as indicated. Nuclei were stained with DAPI. 100× images of z-stacks obtained with a 3i Marianas Inverted Spinning Disc Confocal or 40× images were taken using a standard inverted Nikon fluorescent microscope. FIG. 4B shows TRK-SHC1 PLA applied to cell lines or FFPE tissue demonstrating specificity for activated TRK. These data demonstrate that the TRKA-SHC1 PLA assay is a new assay that can detect activated (oncogenic) TRK in tumor cells and FFPE samples.

The foregoing examples of the present disclosure have been presented for purposes of illustration and description. Furthermore, these examples are not intended to limit the disclosure to the form disclosed herein. Consequently, variations and modifications commensurate with the teachings of the description of the disclosure, and the skill or knowledge of the relevant art, are within the scope of the present disclosure. The specific embodiments described in the examples provided herein are intended to further explain the best mode known for practicing the disclosure and to enable others skilled in the art to utilize the disclosure in such, or other, embodiments and with various modifications required by the particular applications or uses of the present disclosure. It is intended that the appended claims be construed to include alternative embodiments to the extent permitted by the prior art.

1. A method to select a cancer patient who is predicted to respond to the administration of a chemotherapeutic regimen comprising:
detecting in a sample of tumor cells from the patient the presence or absence of a gene fusion selected from the group consisting of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1;
selecting the patient as predicted to respond to the administration of a chemotherapeutic regimen comprising an agent selected from the group consisting of a tyrosine kinase inhibitor, an HSP90 inhibitor, an inhibitor of tyrosine kinase downstream signalling cascade, and combinations thereof if the at least one gene fusion is detected in the sample of tumor cells; or
selecting the patient as predicted to not respond to the administration of a chemotherapeutic regimen comprising an agent selected from the group consisting of a tyrosine kinase inhibitor, an HSP90 inhibitor, an inhibitor of tyrosine kinase downstream signalling cascade, and combinations thereof if the at least one gene fusion is not detected in the sample of tumor cells.

2. The method of claim 1, wherein the detection comprises detecting a level of the at least one gene fusion present in the sample of tumor cells and, comparing the level to a standard level or reference range.

3. The method of claim 2, wherein the standard level or reference range is determined according to a statistical procedure for risk prediction.

4-7. (canceled)

8. The method of claim 1, wherein the detecting of the at least one gene fusion comprises:
obtaining RNA from the sample of tumor cells;
generating cDNA from the RNA;
amplifying the cDNA with primers specific for the at least one gene fusion;
determining the presence or absence of the at least one gene fusion in the amplified cDNA.

9. The method of claim 1, wherein the patient is a human.

10. The method of claim 1, further comprising:
comparing the expression level of the at least one gene fusion in the sample of tumor cells to a control level of the at least one gene fusion selected from the group consisting of:
a) a control level of the at least one gene fusion that has been correlated with beneficial response to the administration of a chemotherapeutic regimen including one or more kinase inhibitor(s); and
a control level of the at least one gene fusion that has been correlated with lack of beneficial response to the administration of a chemotherapeutic regimen including one or more kinase inhibitor(s); and
b) selecting the patient as being predicted to respond to the administration of a chemotherapeutic regimen including one or more kinase inhibitor(s), if the level of the at least one gene fusion in the sample of tumor cells is statistically similar to, or greater than, the control level of the at least one gene fusion that has been correlated with sensitivity to the administration of a chemotherapeutic regimen including one or more kinase inhibitor(s), or
c) selecting the patient as being predicted to not respond to the administration of a chemotherapeutic regimen including one or more kinase inhibitor(s), if the level of the at least one gene fusion in the sample of tumor cells is statistically less than the control level of the at least one gene fusion that has been correlated with beneficial response to the administration of a chemotherapeutic regimen including one or more kinase inhibitor(s).

11. The method of claim 1, further comprising:
comparing the level of the at least one gene fusion in the sample of tumor cells to a level of the at least one gene fusion in a second patient predicted to not respond to the administration of a chemotherapeutic regimen including one or more kinase inhibitor(s), and,
selecting the patient as being predicted to respond to the administration of a chemotherapeutic regimen including one or more kinase inhibitor(s), if the expression level of the at least one gene fusion in the sample of tumor cells is greater than the level of expression of the at least one gene fusion in the second patient, or,
selecting the patient as being predicted to not respond to the administration of a chemotherapeutic regimen including one or more kinase inhibitor(s), if the level of the at least one gene fusion in the sample of tumor cells is less than or equal to the level of the at least one gene fusion in the second patient.

12. The method of claim 1, wherein the tyrosine kinase inhibitor is selected from the group consisting of gefitinib, erlotinib, crizotinib, ponatinib, dovitinib, rebastinib, CEP-701, ARRY-470, RXDX-101, LOXO-101, TSR-011, PLX7486, and combinations thereof.

13. The method of claim 1, wherein the tyrosine kinase inhibitor is a TrkA inhibitor.

14-18. (canceled)

19. A method of diagnosing cancer in a subject, comprising detecting in a sample of tumor cells from the subject the presence of at least one gene fusion selected from the group consisting of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1, wherein the presence of the at least one gene fusion is indicative of cancer in the subject.

20. The method of claim 19, wherein the cancer is selected from a lung cancer, a liver cancer, a colorectal cancer, a thyroid cancer, a melanoma, a glioblastoma, and a glioma.

21. The method of claim 19, wherein the cancer is Non-Small Cell Lung Cancer (NSCLC).

22. The method of claim 19, wherein the presence of the gene fusion is detected by RT-PCR.

23. The method of claim 19, wherein the gene fusion is detected by FISH.

24. The method of claim 23, wherein the presence of the gene fusion is detected by detecting a polypeptide encoded by the NTRK1 gene fusion.

25. The method of claim 24, wherein the polypeptide is detected by using at least one of an antibody, an antibody derivative, and an antibody fragment that specifically binds to the polypeptide, or a fragment thereof.

26. The method of claim 19, wherein the tyrosine kinase inhibitor is selected from the group consisting of gefitinib, erlotinib, crizotinib, ponatinib, dovitinib, rebastinib, CEP-701, ARRY-470, RXDX-101, LOXO-101, TSR-011, PLX7486, and combinations thereof.

27. The method of claim 19, wherein the tyrosine kinase inhibitor is a TrkA inhibitor.

28. (canceled)

29. (canceled)

30. A method for predicting the clinical response of a human lung cancer patient to a tyrosine kinase inhibitor medication comprising:
obtaining a biological sample from a patient diagnosed with a lung cancer, said sample comprising nucleic acids from the patient;
detecting in said nucleic acids the presence of a NTRK1-gene fusion selected from the group consisting of C18ORF8-NTRK1, RNF213-NTRK1, TBC1D22A-NTRK1, C20ORF112-NTRK1, DNER-NTRK1, NELL1-NTRK1, EPL4-NTRK1, CTNND2-NTRK1, and TCEANC2-NTRK1;
wherein said detecting is performed by at least one detection method selected from:
hybridization with a NTRK1 break-apart probe;
Duolink Proximity Ligation Assay (Sigma Aldrich) used with an anti-TRK antibody that recognizes TRKA, TRKB, and TRKC;
an anti-SHC1 antibody;
total nucleic acid sequencing of said nucleic acids using primers corresponding to exons 6, 7, 8, 10, 11, 12, 13, 14 and 15 of NTRK1; and
contacting said nucleic acids with a fluorogenic DNA probe and a DNA polymerase having 5′-exonuclease activity using polymerase chain reaction (PCR); and,
correlating the presence of said NTRK1 gene fusion with an increased likelihood for said patient to have a beneficial clinical response to a tyrosine kinase inhibitor medication.
